(19) United States
(12) Patent Application Publication
(10) Pub. No.: US 2011/0207824 A1
(43) Pub. Date: Aug. 25, 2011

(54) FORMULATION OF AN INJECTABLE PARACETAMOL SOLUTION, METHOD FOR PREPARING AND PACKAGING SUCH A SOLUTION AND DEVICE FOR PACKAGING SUCH A SOLUTION

(75) Inventor: Didier DOULEAU, Cambernard (FR)

(73) Assignee: MOLY PHARMA, Cambernard (FR)

(21) Appl. No.: 12/712,412

(22) Filed: Feb. 25, 2010

(51) Int. Cl. A61K 31/167; B65B 55/02; B65B 63/08; B65B 3/04; A61J 1/14

(52) U.S. Cl. 514/629; 53/425; 53/428; 53/440; 53/469; 206/570; 206/438

(57) ABSTRACT

A simplified paracetamol solution for an injectable and ready-to-use preparation is only composed of paracetamol, bi-distilled water and a buffer agent, the formulation having a maximal paracetamol concentration of 1 g/100 ml, a pH between 5.5 and 6.5 and an oxygen content inferior to 0.2 ppm. A method for preparing and packaging such a solution and a device for packaging such a solution are also disclosed.

The present invention relates to a simplified paracetamol solution for an injectable and ready-to-use preparation. According to the invention, said paracetamol solution is only composed of paracetamol, bi-distilled water and a buffer agent. The invention also relates to a method for preparing and packaging such a solution and a device for packaging such a solution.

Paracetamol, like many active ingredients, is sensitive to the presence of oxygen in the air as well as to oxygen dissolved in conventional aqueous solvents used for preparing the injectable solution. Besides, the stability of the solution depends upon pH conditions of the medium which can vary during the time because of the reaction with some chemical compounds in the composition of packaging articles for the solution.

The chemical degradation of paracetamol goes through the formation of 4-paraminophenol, 3-paraminophenol which are converted into colored quinoid derivatives. The degradation of paracetamol can result in some dimerized forms of paracetamol.

In order to overcome this problem, several methods have been used which consist in:
neutralizing the oxygen dissolved in the solution by adding to it some antioxidant agents such as ascorbic agent or cysteine;
eliminating the dissolved oxygen by splashing an inert gas, such as nitrogen or its dioxide, in the solution;
stabilizing the solution pH by mixing buffers about 6.

All these prior methods have some effectiveness, but the packaged desoxygenated solutions always contain a residual quantity of dissolved oxygen.

The paracetamol solutions of the state of the art can have some irritating or allergenic effects due to the presence of antioxidant agents.

Besides, it is known that some antioxidants can become degraded due to heat. For that reason, it is essential to add some compounds such as divalent metallic salts for maintaining the antioxidant integrity.

Thus, the existing paracetamol formulas in injectable solution are composed of many additives which make the solution preparation complex and expensive, because there are a lot of opportunities for oxygen to be incorporated with materials into the solution although precautions are taken to avoid it.

Otherwise, in order to ensure the stability of the packaged solution, the packaging devices proposed are generally glass flasks plugged with elastomeric plugs.

The solutions can also be packaged in flexible pockets plugged with elastomeric plugs. Packaging in flexible pocket can ensure a handling security in health care centers as regards the risk of introducing air into the solution when the product is administered, which can result in hospital-acquired infections and air embolisms.

As far as the filling technique for these both packaging types is concerned, the glass flask requires maintaining an air space between the liquid and the plug in order to enable the sterilization operation and to avoid the explosion of the flask or the accidental opening thereof during this operation. This header space must be in the order of 20 or 30 ml and must be filled with an inert gas or evacuated.

The flexible pocket is advantageous in that it enables to fill more the effective volume and to reduce the header space to a volume in the order of 5 to 10 ml without needing to treat the gaseous content in this space. Besides, the plastic material enables to sterilize the pockets without any risk of explosion or fissuring.

However, all these prior packaging devices proposed mainly aim at protecting the solution from the external environment and they do not enable to solve the oxidation problem due to the oxygen dissolved in the solution.

The main objective of the present invention is protecting the paracetamol solution from later oxidation by the oxygen dissolved in the solution due to operations for preparing the solution.

In order to overcome the above-described technical problems, the concept of the present invention is based on:
the simplified formulation of a paracetamol solution wherein the number of raw material components is reduced in order to enable the optimization of the operations and the preparation time;
a method of automatic preparation which includes the continuous checking of parameters connected with the quality of the solution such as oxygen content, paracetamol titer, and pH;
a packaging device including an oxygen absorbent which enables to catch the oxygen dissolved in the paracetamol solution.

For that purpose, the invention relates to a formulation of an injectable and ready-to-use paracetamol solution.

According to the invention, the formulation of the paracetamol solution is only composed of paracetamol, bi-distilled water and a buffer agent, said formulation having a maximal concentration of paracetamol of 1 g/100 ml, a pH between 5.5 and 6.5, and an oxygen content inferior to 0.2 ppm.

Such a simplified formulation enables the optimization of the operations and of the preparation time for the solution. It enables to save manufacturing costs in comparison with the existing formulations. Besides, the reduced number of raw materials in the formulation enables to integrate all the preparation operations into a unique hermetically sealed reactor and thus to eliminate the risks of introducing oxygen and impurities during the preparation operations.

Preferably, the selected buffer agent is an acetate buffer agent which provides in the formulation according to the present invention only chemical components identical to the acetyl radical of the paracetamol molecule.

The acetate buffer mixture enables to stabilize the solution pH between 5.5 and 6.5 and preferably at pH 6 in order to optimize the paracetamol stability and to authorize a solution packaging in flexible PVC pocket which can result, during the time, in a diminution of the solution pH and influence the stability of the active principle.

Preferably, this acetate buffer mixture is composed of sodium trihydrate acetate and of glacial acetic acid.

The invention relates to a method for preparing and packaging a paracetamol solution, such as described above, by means of a hermetically sealed reactor, said reactor being composed of a vessel hermetically connected to a bi-distilled water distribution circuit, an injection circuit for white vapor of bi-distilled water, a dissolution circuit for paracetamol and sodium acetate, a micro-dosing apparatus for glacial acetic acid and a solution transferring circuit.

According to the invention, the method comprises the following steps of:

a) introducing a volume of bi-distilled water into the vessel through the bi-distilled water distribution circuit, said vessel being first cleaned with white vapor of bi-distilled water;

b) cooling the volume of water until a temperature between 30° C. and 35° C. is achieved;

c) checking the oxygen content dissolved in the volume of water and realizing a desoxygenation by means of the white vapor of bi-distilled water if the oxygen content is superior to 0.2 ppm;

d) introducing by aspiration paracetamol and sodium acetate in a powder form into said dissolution circuit, paracetamol and sodium acetate being then dissolved in the dissolution circuit by means of a ultra-dispersing machine;

e) returning the mixture obtained at step d) into the vessel through the transferring circuit;

f) adjusting the solution pH with the micro-dosing apparatus for glacial acetic acid and complementing the final volume of the mixture with bi-distilled water;

g) checking the oxygen content of the solution and realizing a desoxygenation by means of the white vapor if the oxygen content is superior to 0.2 ppm;

h) transferring the paracetamol solution through a first 0.45 μm-filter then through a second 0.22 μm-filter;

i) realizing the packaging step consisting in filling a flexible pocket with the paracetamol solution filtered at the step h), and in hermetically packaging said pocket into an over-package with an oxygen absorbent; and

the steps a) to h) being realized in said hermetic reactor in an atmosphere desoxygenated with the white vapor of bi-distilled water.

Thus, this approach consisting in simplifying the formulation of the above-described paracetamol solution has enabled to develop a simplified industrial method, wherein various preparation operations are combined in only one preparation device, called here hermetically sealed reactor, and also in integrating a continuous checking of parameters connected with the quality of the solution such as oxygen content and pH during the preparation steps. Thus, this continuous checking enables to adjust parameters, if needed, in order that the final solution complies with the optimal stability conditions.

Another technical advantage relates to the operating mode of the method which uses bi-distilled water as a formulation component and white vapor of bi-distilled water as:
an inerting agent for the solution during the preparation, if needed;
a transferring gas during the filling operation for the vessel and the draining operation at the end of the preparation;
a cleaning agent for the preparation device between two production series.

Preferably, the solid materials, which are paracetamol and sodium acetate introduced into the dissolution circuit, will have been stored before their use in a hermetically sealed bag and desoxygenated in the presence of an oxygen getter.

The invention also relates to a packaging device for an injectable and ready-to-use paracetamol solution.

According to the invention, it comprises:
a primary package consisting in a flexible pocket composed of at least one compartment, one of the compartments containing said solution,
a secondary package used as an over-package for said pocket and comprising two material walls, the edges of which are sealed for ensuring the tightness properties of the over-package,
an oxygen absorbent placed in said over-package, said absorbent being destined to catch the oxygen dissolved in the solution and the oxygen present in the over-package.

One of the main advantages of the packaging device, which has just been defined, lies in the combination of three package components: the pocket, the over-package and the absorbent which will initiate a diffusion reaction for the oxygen dissolved in the paracetamol solution, packaged in the pocket, through the pocket walls and the oxygen absorption by chemical getters.

Other characteristics and advantages of the invention will become apparent from the following description in reference to the annexed schematic drawings which are given as examples only and in which:

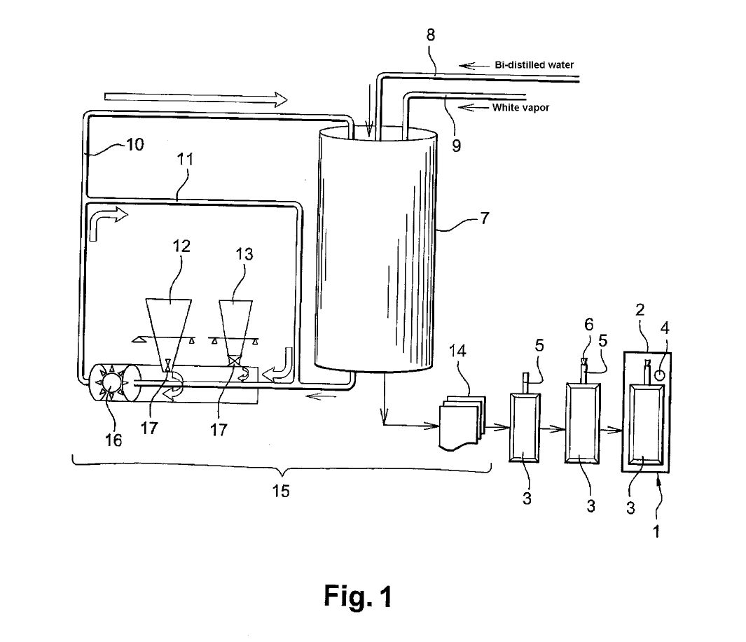

FIG. 1 is a schematic representation of the various steps of the method for preparing and packaging a paracetamol solution according to the invention;

FIG. 2 is a schematic representation of the packaging device for an injectable and ready-to-use paracetamol solution according to the invention;

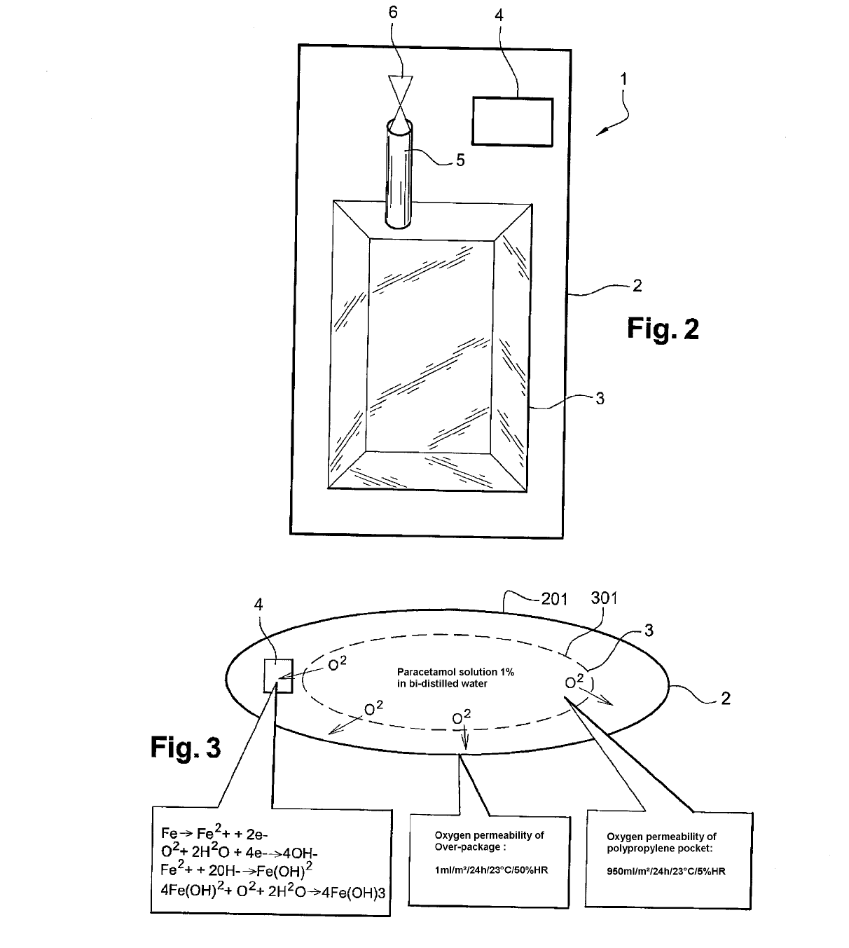

FIG. 3 is a schematic representation of the oxygen absorption process by the oxygen absorbent.

The following table shows the various components of the simplified formulation of the paracetamol solution according to the invention:

| Composition | Function |
|---|---|
| Paracetamol | Active principle |
| Sodium trihydrate acetate | Buffer agent |
| Glacial acetic acid | Buffer agent |
| Bi-distilled water | Dissolution and dilution solvent |

FIG. 1 represents the method for preparing such a simplified formulation of the paracetamol solution, wherein the operations of weighing the components, of dispersing and dissolving and filtrating are integrating in only one hermetically sealed device as regards the external environment.

This device, more generally called reactor 15, is composed of a vessel 7 hermetically connected to a bi-distilled water distribution circuit 8, an injection circuit 9 for white vapor of bi-distilled water, a dissolution circuit 11 for paracetamol and sodium acetate and a solution transferring circuit 10.

Advantageously, this device is provided at the vessel 7 with means, not represented in FIG. 7, for continuously checking various solution parameters such as oxygen content, paracetamol titer, and pH. The measured values can be periodically sent to a graphic interface or transferred only when demanded by an operator.

The method of the present invention can be applied to reactors with a capacity from 100 liters to 5000 liters.

The method comprises the following steps:

Step 1:

The vessel 7 is filled with bi-distilled water used as dissolution and dilution solvent through the bi-distilled water distribution circuit 8. This vessel is first filled with white vapor injected through the circuit 9 in order to eliminate any air traces. Preferably, the vessel is filled with bi-distilled water in a volume equivalent to 90% of the volume of the final mixture.

Step 2:

The bi-distilled water is then cooled until an adapted temperature between 30° C. and 35° C. is achieved, preferably 30° C. The content of oxygen dissolved in the volume of water is then checked and a desoxygenation is performed by means of the white vapor if the measured content is superior to 0.02 ppm.

Step 3:

The step of dissolution of the solid raw materials is performed according to the following operations:
the dissolution circuit 11 is supplied with bi-distilled water from the vessel 7 by starting the high-flow pump 16,
the paracetamol and sodium acetate stored in a powder form in the tanks 12, 13 are automatically weighed.
the weighed materials are sucked through a pipe 17 connected to an ultra-disperser 16 which performs a high-speed dispersion of the powders in water and then a dissolution of the products in the circuit 11.

Step 4:

After dissolution of the raw materials in the circuit 11, this circuit is connected to the transferring circuit 10 for transferring the paracetamol solution into the final mixture vessel 7. The pH is checked and then adjusted to 6 by automatically supplying acetic acid and the final volume of the mixture will be adjusted by supplying bi-distilled water.

The oxygen content is checked and adjusted again to a value at least inferior to 0.2 ppm. The final mixture is maintained while stirring until the conformity is checked.

Step 5:

Before packaging the solution, it is submitted to a first filtration 14 with a 0.45 μm-filter and then to a second filtration with a 0.22 μm-filter, which is a sterilizing filtration.

All these above-described operations are performed in an atmosphere desoxygenated with white vapor of bi-distilled water.

Step 6:

The last step of the method relates to the packaging of the final solution which comprises the following operations:
a flexible pocket 3 is filled with the filtered paracetamol solution; in the case that the flexible pocket comprises two compartments, one of the compartments is filled with the solution as the same as the second compartment is also filled with another component such as codein or Ketoprofe; once filling is achieved, the tubular aperture 5 of the pocket is plugged with a plug 6 such as a quarter-turn cap;
the flexible pocket 3 is then packaged into an over-package 2 with an oxygen absorbent 4 and the over-package is sealed in order to ensure the tightness of the whole.

Preferably, the packaging step for the solution is performed in a room with a cleanliness class A or B (3500 particles/m³) so as to avoid the autoclaving sterilization step.

In the case that the packaging step is performed in a room with a cleanliness class C, the method comprises a supplemental step consisting in an autoclaving sterilization of the packaged solution, i.e. the solution is heated at 121° C. for 15 min.

FIG. 2 represents a preferred embodiment of the device for packaging the paracetamol solution. It comprises a flexible pocket 3 in which the paracetamol solution is packaged. In order to protect the solution from the external air, the pocket is placed in an over-package 2 with oxygen absorbent 4.

Generally, this oxygen absorbent is in the form of a bag or cap containing a mixture of metallic-oxides-based mineral material, preferably iron-oxide-based mineral material with no toxicity for eating use.

In order to ensure the efficiency of the packaging device and to increase the solution life, a partial vacuum is realized in the space between the pocket and the over-package before sealing the over-package.

The over-package is preferably made from a complex material polyester/polyamide/aluminum/polypropylene which is thus opaque. However, the over-package can also be made from a complex material polyester/polypropylene, and the results of the below-described study III show that the stability of the solution is not affected by the use of such a material. Preferably, the walls of the over-package have an oxygen permeability which is at least inferior to 1 cm³/m²/24 h at T=23° C. and in a relative ambient humidity of 50% HR.

The flexible pocket is made from a plastic material selected from a group comprising polyvinyl chloride, polypropylene or a polypropylene-based multilayer complex material. Preferably, the pocket walls have an oxygen permeability between 950 cm³/m²/24 h and 1000 cm³/m²/24 h at T=23° C. and in a relative ambient humidity of 50% HR.

The antioxidant material, contained in the bag in the presence of a low humidity level, will initiate a chemical fixation reaction for the gaseous oxygen in the environment. The absorption capacity is adjustable and is between 100 and 400 ml of oxygen.

FIG. 3 represents the chemical reaction principle which is as follows:

Fe→Fe²+2e⁻

O²+2H²O+4e⁻→OH⁻

Fe²⁺+2OH⁻→Fe(OH)²

4Fe(OH)²+O²+2H²O→4Fe(OH)³

According to the innovation the getter 4 is located between the wall of the pocket containing the paracetamol solution and the wall of the over-package as represented in FIG. 2. The gas permeability difference, notably for the oxygen between the walls 301 of the flexible pocket 3 and the walls 201 of the over-package 2, will result in the transfer of the dissolved oxygen from the solution to the medium containing the absorbent 4.

The following description relates to the results of three studies performed with prototypes of solutions prepared according to the method as described above.

Study I: Checking of the Feasibility of the Formula and of its Packaging with or without Oxygen Getter

The following table sums up the main measure parameters during the various operations for preparing the solution:

| Operation | Tests | Results |
|---|---|---|
| Reactor supply with bi-distilled water | Volume | 89 liters |
| | Temperature | 75° C. |
| | Cooling | 31° C. |
| | Oxygen ratio | 0.07 mg/l |
| Paracetamol incorporation | Dissolution time | 10 minutes |
| | Limpidity | Limpid |
| | | solution |
| Final volume adjustment with bi- | Volume | 11 liters |
| distilled water | | |
| pH adjustment | pH measure | 6.0 |
| | Sodium acetate | |
| | quantity | |
| Final pH adjustment with acetic acid | Oxygen ratio | 0.04 mg/l |
| | Density | 1.0069 |
| Filtration 0.22 μm | Particle control | 0 |
| Polypropylene Pocket Filling | Volume Control | 100 ml |

In this test, the final mixture of 100 liters has been packaged in polypropylene pockets of 100 ml by using semi-automatic industrial devices located in a pharmaceutical zone of class C without any protection from laminar flow of inert gas.

Once they were filled, these pockets were separated in two secondary packaging sets:
Sets 10788-01 (over-packaging of the pocket without any oxygen absorbent)
Sets 10788-02 (over-packaging of the pocket with an oxygen absorbent having an absorption capacity of 400 ml)

The material used for the over-package is the same for both sets, i.e. a complex material polyester/polyamide/aluminum/polypropylene.

The material of the oxygen absorbent is a mixture of mineral oxides.

These two sets were analyzed, sterilized at 121° C. for 15 minutes, and then stabilized after a new analytic checking according to the following protocol:

a) Storage temperature: 55° C. Period: 30 days. Periodicity: T0, T+15, T+30
b) Storage temperature: 40° C. Period: 90 days. Periodicity: T0, T+30, T+60, T+90
c) Storage temperature: 25° C. Period: 9 months. Periodicity: T0, T+6 months, T+9 months

The characteristics observed for showing the quality of the method formula and the package were: the paracetamol titer, the colored aspect of the solution, the pH, and the dosage of total impurities.

We chose the coloration index as a discriminating factor of these tests, because the destabilization of Paracetamol in the presence of oxygen goes through the apparition of instable paraminophenol which is converted into colored quinoid derivatives.

The second discriminating criterion concerns the titer in total impurities in comparison with Paracetamol, but during the early months of our test we were not able to check these impurities through lack of relevant and validated analytic method. It is only from the third month that this second criterion was integrated into our analysis.

The following table shows the below-stated conclusions:

| | | | | Paracetamol |
|---|---|---|---|---|
| | | Total | pH | titer |
| | Coloration | impurities | 5.5 | 0.950 g/100 ml |
| | (absorbance | in % of | to | To |
| Determination | at 430 nm) | paracetamol | 6.5 | 1.050 g/100 ml |
| Pocket before | 0 | / | 6.0 | 0.986 |
| autoclave | | | | |
| T0 with no getter | 0.009 | / | 6.0 | 1.023 |
| after autoclave | | | | |
| T0 with getter | 0.009 | / | 6.0 | 1.023 |
| after autoclave | | | | |
| 55° C. T + 15 | 0.019 | / | 6.0 | 1.003 |
| with no getter | | | | |
| 55° C. T + 15 | 0.007 | / | 6.0 | 1.014 |
| with getter | | | | |
| 55° C. T + 30 | 0.029 | / | 6.0 | 0.988 |
| with no getter | | | | |
| 55° C. T + 30 | 0.006 | / | 6.0 | 0.999 |
| with getter | | | | |
| 40° C. T + 30 | 0.013 | / | 6.0 | 0.985 |
| with no getter | | | | |
| 40° C. T + 30 | 0.007 | / | 6.0 | 0.991 |
| With getter | | | | |
| 40° C. T + 60 | 0.020 | / | 6.0 | 1.022 |
| with no getter | | | | |
| 40° C. T + 60 | 0.007 | / | 6.0 | 1.034 |
| with getter | | | | |
| 40° C. T + 90 | 0.025 | 0.095 | 6.0 | 1.007 |
| with no getter | | | | |
| 40° C. T + 90 | 0.007 | 0.094 | 6.0 | 1.006 |
| with getter | | | | |
| 25° C. T + 6 months | 0.014 | 0.134 | 6.0 | 1.030 |
| with no getter | | | | |
| 25° C. T + 6 months | 0.010 | 0.128 | 6.0 | 1.040 |
| with getter | | | | |
| 25° C. T + 9 months | 0.019 | 0.152 | 6.0 | 1.020 |
| with no getter | | | | |
| 25° C. T + 9 months | 0.009 | 0.134 | 6.0 | 1.040 |
| with getter | | | | |

Considering the results of the stability study, it clearly appears that:
the simplified formula of paracetamol is stable in the most hard temperature conditions, the variations of the paracetamol content are included in the variability of the analytic method;
an important difference in coloration index is in favor of solutions packaged in over-packages provided with oxygen getters. The phenomenon can be explained by the difference in impurity content 4-aminophenol founded in both pocket categories. The pockets with getter contain paraminophenol stabilized by the absence of oxygen, whereas the pockets with no getter have no more paraminophenol, which was transformed by the presence of uncaught oxygen;
it is noted that, after a period of 90 days at the temperature of 40° C., the percentage of total impurities is the same between pockets with oxygen getters and pockets with no getters;
it is noted that, after a 9 months stabilization at 25° C., an important difference in impurity content appears between both sets of pockets and in favor of pockets with oxygen getters;
the pH remains stable whatever the studied conditions.

The results of this test unexpectedly show that the stability of the ready-to-use paracetamol solution according to the present invention can be obtained in the absence of excipient, unlike the solutions of the prior art. Paracetamol can be stabilized by the simplified formula, a preparation method integrating all the preparation operations in a hermetically sealed device as regards the external environment as well as a packaging device provided with an absorbent.

### Study II: Stability of the Solution According to pH

The second test aims at proving that pH is a critical parameter for the elaboration of the formula, and that pH 6 ensures a good stability of the formula.

The test protocol consists in injecting into the pockets a solute PROTOTYPE-1 which has been first stored at the ambient temperature, doses of hydrochloric acid or of soda for obtaining pockets the pH of which is between 4.5 and 7.5. After injecting acidifying agents or alkalinizing agents, the aperture realized by the needle in the plug is closed up by the materials used for the production of perfusion pocket plugs.

These samples were placed in an oven at 55° C. for 30 days and were analyzed 15 days later and 30 days later.

| | | Total | | |
|---|---|---|---|---|
| | Coloration | impurities | | Paracetamol |
| | (absorbance | in % of | | titer |
| Determination | at 430 nm) | paracetamol | pH | (g/100 ml) |
| pH 4.5 T + 15 days | 0.071 | 0.266 | 4.51 | 1.02 |
| pH 4.5 T + 30 days | 0.139 | 0.284 | 4.55 | 1.05 |
| pH 5.5 T + 15 days | 0.043 | 0.243 | 5.44 | 1.02 |
| pH 5.5 T + 30 days | 0.067 | 0.319 | 5.44 | 1.05 |
| pH 6 T + 15 days | 0.035 | 0.331 | 6.03 | 1.01 |
| pH 6 T + 30 days | 0.052 | 0.435 | 6.02 | 1.05 |
| pH 6.5 T + 15 days | 0.046 | 0.483 | 6.51 | 1.04 |
| pH 6.5 T + 30 days | 0.059 | 0.667 | 6.47 | 1.04 |
| pH 7.5 T + 15 days | 0.107 | 0.898 | 7.21 | 1.05 |
| pH 7.5 T + 30 days | 0.121 | 1.534 | 7.00 | 1.02 |

It can be noted that, beyond pH 6.5, it is observed an important degradation indicated by a very strong coloration of the solution as well as by an important impurity ratio.

In the acidity zones, i.e. at pH 4.5, it is observed a very strong coloration but a very low impurity level.

### CONCLUSION

Our choice of buffer pH 6 is the best compromise of the series with the lowest coloration index and the slowest evolution of the impurity ratio.

### Study III: Influence of the Over-Package Materials on the Solution Stability

The third study aims at checking the technical feasibility of using materials Polyester/Polypropylene for producing the over-package. It is true that, in addition to their tightness to gases and notably to oxygen, they are transparent, which enables to ensure a visual checking after the over-packaging process during the production but also when they are used by nurses.

For this test, we have reduced the absorption capacity from 400 ml to 100 ml of oxygen for checking the permeability of the perfusion pocket and the behavior of the paracetamol solution. The set dimension is of 100 liters and it was packaged without any particular protection conditions in inert gas in three sets:
Set 1 over-packaged in an aluminum complex with oxygen getter (100 ml),
Set 2 over-packaged in an aluminum complex with no getter,
Set 3 over-packaged in a polyester/polypropylene complex with oxygen getter (100 ml).

After sterilization at 121° C. for 15 minutes, these three sets were placed in an oven at 55° C. during one month and analyzed with the same protocols as those used in the first study and the discriminating parameters were the coloration of the solution and the total impurity ratio.

The following table shows the above-stated conclusions:

| | | Total | | |
|---|---|---|---|---|
| | Coloration | impurities | | Paracetamol |
| | (absorbance at | in % of | | titer |
| Determination | 430 nm) | paracetamol | pH | (g/100 ml) |
| Complex | | | | |
| Aluminum | | | | |
| with getter | | | | |
| 55° C. T − 0 | 0.004 | 0.07% | 5.92 | 1.03 |
| 55° C. T + 7 | 0.004 | 0.06% | 5.90 | 1.01 |
| 55° C. T + 15 | 0.003 | 0.06% | 5.89 | 1.03 |
| 55° C. T + 30 | 0.004 | 0.06% | 5.90 | 1.03 |
| Complex Aluminum | | | | |
| with no getter | | | | |
| 55° C. T − 0 | 0.004 | 0.07% | 5.92 | 1.03 |
| 55° C. T + 7 | 0.004 | 0.08% | 5.91 | 1.01 |
| 55° C. T + 15 | 0.015 | 0.09% | 5.90 | 1.02 |
| 55° C. T + 30 | 0.030 | 0.13% | 5.90 | 1.03 |
| Complex | | | | |
| polypropylene | | | | |
| with getter | | | | |
| 55° C. T − 0 | 0.004 | 0.06% | 5.90 | 1.03 |
| 55° C. T + 7 | 0.006 | 0.07% | 5.92 | 1.02 |
| 55° C. T + 15 | 0.005 | 0.06% | 5.90 | 1.02 |
| 55° C. T + 30 | 0.008 | 0.07% | 5.90 | 1.05 |

The examination of this test confirms the interest of the oxygen getter in ensuring the absorption of oxygen dissolved in paracetamol and shows that it is possible to increase the preservation security for the solution by means of getters the absorption capacity of which is in a range from 100 ml to 400 ml of oxygen.

This test demonstrates that it is possible to use a transparent material for the over-package instead of aluminum complexes provided that the difference in gas tightness between the perfusion pocket and the over-package bag is in the order of 900.

The following tables show the results of an accelerated stability study between a simplified paracetamol solution obtained according to the invention and a marketed solution such as Perfalgan.

Impurity K=4 aminophenol, NP: Not Realized, ND: Not Detected, RT: Report Threshold (0.05%)

| STORAGE | Initial time | Paracetamol 1 g IV | | | Marketed solution | | |
|---|---|---|---|---|---|---|---|
| AT +55° C. | | Pocket for perfusion | | | | | |
| Package Description | | Flexible 100 ml-pocket over- | | | Glass 100 ml-Bottle plugged | | |
| | | packaged in an aluminum | | | with an elastomeric plug | | |
| | | pocket with oxygen absorber | | | | | |
| | Specifications for | | | | | | |
| DETERMINATIONS | the inventive solution | 801101 | 801401 | 801402 | 7G1301 | 8D34475 | 8F34140 |
| PHARMACEUTICAL | | | | | | | |
| TESTS | | | | | | | |
| Nature | Limpid and not | Conform | Conform | Conform | Conform | Conform | Conform |
| | more colored | | | | | | |
| | than J5 and R6 | | | | | | |
| Coloration at | For information | 0.005 | 0.004 | 0.007 | 0.003 | 0.003 | 0.001 |
| 430 nm | | | | | | | |
| TESTS | | | | | | | |
| pH | 5.5-6.5 | 6.0 | 6.0 | 6.0 | 5.6 | 5.6 | 5.7 |
| Density | | 1.002 | 1.007 | 1.005 | 1.014 | NP | 1.015 |
| Osmolality | 270.0-310.0 | 288.0 | 293.0 | 288.7 | 291.7 | NP | 292.0 |
| (mOsm/kg) | | | | | | | |
| DOSAGE | | | | | | | |
| Paracetamol | 9.50-10.50 | 10.00 | 10.29 | 10.23 | 10.08 | 9.79 | 9.84 |
| (mg/ml) | | | | | | | |
| PURITY TESTS (%) | | | | | | | |
| Impurities K | ≦0.05 | 0.01 | 0.01 | 0.01 | 0.01 | 0.02 | 0.02 |
| Dimeric | ≦0.05 | 0.01 | 0.01 | 0.01 | ND | ND | ND |
| Paracetamol | | | | | | | |
| Individual | ≦0.10 | <RT | <RT | <RT | <RT | <RT | <RT |
| unknown | | | | | | | |
| impurity | | | | | | | |
| Total unknown | ≦0.10 | <RT | <RT | <RT | <RT | <RT | <RT |
| impurities | | | | | | | |

| STORAGE | T + 14 days | Paracetamol 1 g/100 ml | | | Marketed solution | | |
|---|---|---|---|---|---|---|---|
| AT +55° C. | | Pocket for perfusion | | | | | |
| Package Description | | Flexible 100 ml-pocket over- | | | Glass 100 ml-Bottle plugged | | |
| | | packaged in an aluminum | | | with an elastomeric plug | | |
| | | pocket with oxygen absorber | | | | | |
| | Specifications for | | | | | | |
| DETERMINATIONS | the inventive solution | 801101 | 801401 | 801402 | 7G1301 | 8D34475 | 8F34140 |
| PHARMACEUTICAL | | | | | | | |
| TESTS | | | | | | | |
| Nature | Limpid and not | NP | Conform | Conform | Conform | Conform | Conform |
| | more colored | | | | | | |
| | than J5 and R6 | | | | | | |
| Coloration at | For information | NP | 0.004 | 0.007 | 0.001 | 0.002 | 0.001 |
| 430 nm | | | | | | | |
| TESTS | | | | | | | |
| pH | 5.5-6.5 | NP | 6.0 | 6.0 | 5.6 | 5.4 | 5.7 |
| Density | | NP | 1.007 | NP | 1.013 | NP | 1.014 |
| Osmolality | 270.0-310.0 | NP | 295.0 | NP | 294.7 | NP | 288.7 |
| (mOsm/kg) | | | | | | | |
| DOSAGE | | | | | | | |
| Paracetamol | 9.50-10.50 | NP | 10.33 | 10.3 | 10.15 | 9.91 | 9.86 |
| (mg/ml ) | | | | | | | |
| PURITY TESTS (%) | | | | | | | |
| Impurities K | ≦0.05 | NP | 0.03 | 0.02 | 0.03 | 0.04 | 0.03 |
| Dimeric | ≦0.05 | NP | 0.01 | 0.01 | ND | ND | ND |
| Paracetamol | | | | | | | |
| Individual | ≦0.10 | NP | <RT | <RT | <RT | <RT | <RT |
| unknown | | | | | | | |
| impurity | | | | | | | |
| Total unknown | ≦0.10 | NP | <RT | <RT | <RT | <RT | <RT |
| impurities | | | | | | | |

| STORAGE | T + 21 days | Paracetamol 1 g/100 ml | | | Marketed solution | | |
|---|---|---|---|---|---|---|---|
| AT +55° C. | | Pocket for perfusion | | | | | |
| Package Description | | Flexible 100 ml-pocket over- | | | Glass 100 ml-Bottle plugged | | |
| | | packaged in an aluminum | | | with an elastomeric plug | | |
| | | pocket with oxygen absorber | | | | | |
| | Specifications for | | | | | | |
| DETERMINATIONS | the inventive solution | 801101 | 801401 | 801402 | 07G1301 | 8D34475 | 8F34140 |
| PHARMACEUTICAL | | | | | | | |
| TESTS | | | | | | | |
| Nature | Limpid and not | Conform | Conform | Conform | Conform | Conform | Conform |
| | more colored | | | | | | |
| | than J5 and R6 | | | | | | |
| Coloration at | For information | 0.004 | 0.007 | 0.015 | Not | 0.014 | 0.001 |
| 430 nm | | | | | measurable | | |
| TESTS | | | | | | | |
| pH | 5.5-6.5 | 6.0 | 6.0 | 6.0 | 5.5 | 5.3 | 5.6 |
| Density | | 1.008 | 1.008 | NP | 1.016 | NP | 1.015 |
| Osmolality | 270.0-310.0 | 285.0 | 294.5 | NP | 295.5 | NP | 289.5 |
| (mOsm/kg) | | | | | | | |
| DOSAGE | | | | | | | |
| Paracetamol | 9.50-10.50 | 10.03 | 10.18 | 10.27 | 10.04 | 9.85 | 9.74 |
| (mg/ml) | | | | | | | |
| PURITY TESTS (%) | | | | | | | |
| Impurities K | ≦0.05 | 0.03 | 0.03 | 0.02 | 0.03 | 0.04 | 0.03 |
| Dimeric | ≦0.05 | 0.02 | 0.01 | 0.01 | ND | ND | ND |
| Paracetamol | | | | | | | |
| Individual | ≦0.10 | <RT | <RT | <RT | <RT | <RT | <RT |
| unknown | | | | | | | |
| impurity | | | | | | | |
| Total unknown | ≦0.10 | <RT | <RT | <RT | <RT | <RT | <RT |
| impurities | | | | | | | |

| STORAGE | T + 3 months | Paracetamol 1 g/100 ml | | | Marketed solution | | |
|---|---|---|---|---|---|---|---|
| AT +55° C. | | Pocket for perfusion | | | | | |
| Package Description | | Flexible 100 ml-pocket over- | | | Glass 100 ml-Bottle plugged | | |
| | | packaged in an aluminum | | | with an elastomeric plug | | |
| | | pocket with oxygen absorber | | | | | |
| | Specifications for | | | | | | |
| DETERMINATIONS | the inventive solution | 801101 | 801401 | 801402 | 7G1301 | 8D34475 | 8F34140 |
| PHARMACEUTICAL | | | | | | | |
| TESTS | | | | | | | |
| Nature | Limpid and not | Conform | Conform | Conform | Conform | Conform | Conform |
| | more colored | | | | | | |
| | than J5 and R6 | | | | | | |
| Coloration at | For | 0.006 | 0.005 | 0.012 | Not | Not | 0.002 |
| 430 nm | information | | | | measurable | measurable | |
| TESTS | | | | | | | |
| pH | 5.5-6.5 | 6.0 | 6.0 | 6.0 | 5.4 | 5.3 | 5.6 |
| Density | | 1.007 | 1.007 | 1.006 | 1.012 | 1.016 | 1.013 |
| Osmolality | 270.0-310.0 | 283.0 | 290.0 | 289.3 | 293.0 | 284.3 | 285.0 |
| (mOsm/kg) | | | | | | | |
| DOSAGE | | | | | | | |
| Paracetamol | 9.50-10.50 | 9.86 | 10.10 | 10.17 | 9.89 | 9.79 | 9.58 |
| (mg/ml) | | | | | | | |
| PURITY TESTS (%) | | | | | | | |
| Impurities K | ≦0.05 | 0.06 | 0.07 | 0.08 | 0.11 | 0.16 | 0.08 |
| Dimeric | ≦0.05 | <0.01 | 0.01 | 0.01 | ND | ND | ND |
| Paracetamol | | | | | | | |
| Individual | ≦0.10 | <RT | <RT | <RT | <RT | <RT | <RT |
| unknown | | | | | | | |
| impurity | | | | | | | |
| Total unknown | ≦0.10 | <RT | <RT | <RT | <RT | <RT | <RT |
| impurities | | | | | | | |

This comparative stability study between a paracetamol solution of 1 g and a perfalgan solution shows two main differences:
the impurity ratio of 4-aminophenol increases much faster in the currently marketed solution such as Perfalgan, and, after 3 months storage, the mean ratio obtained in this conventional solution is twice higher than the mean ratio obtained in the paracetamol solution of 1 g prepared according to the invention,
a low presence of dimeric paracetamol in the paracetamol solution of 1 g prepared according to the invention, the ratio does not however increase with the storage duration,
it appears from the results that there is a small pH variation in the solution of the prior art, whereas, for the same storage duration, the paracetamol solution prepared according to the invention has a good pH stability.

For both studied products, the physicochemical parameters such as the density and the osmolality show no variation and the active ingredient content is stable. The unknown impurity ratio does not increase during the storage and it remains very low (about 0.02% for both products).

1. Formulation for an injectable and ready-to-use paracetamol solution, wherein said paracetamol solution is only composed of paracetamol, bi-distilled water and a buffer agent, said formulation having a maximal concentration of paracetamol of 1 g/100 ml, a pH between 5.5 and 6.5, and an oxygen content inferior to 0.2 ppm.

2. Formulation according to claim 1, wherein the buffer agent is a buffer agent couple composed of sodium acetate and of acetic acid.

3. Formulation according to claim 1, wherein the solution pH is 6.

4. Formulation according to claim 1, wherein the quantity of paracetamol is 1 g/100 ml.

5. Method for preparing and packaging a paracetamol solution according to claim 1 by means of a hermetically sealed reactor, said reactor being composed of a vessel hermetically connected to a bi-distilled water distribution circuit, an injection circuit for white vapor of bi-distilled water, a dissolution circuit for paracetamol and sodium acetate and a solution transferring circuit, said method further comprising the following steps of:
a) introducing a volume of bi-distilled water into the vessel through the bi-distilled water distribution circuit, said vessel being first cleaned with white vapor of bi-distilled water;
b) cooling the volume of water until a temperature between 30° C. and 35° C. is achieved;
c) checking the oxygen content dissolved in the volume of water and realizing a desoxygenation by means of white vapor of bi-distilled water if the oxygen content is superior to 0.2 ppm;
d) introducing by aspiration paracetamol and sodium acetate in a powder form into said dissolution circuit, paracetamol and sodium acetate being then dissolved in the dissolution circuit by means of a ultra-dispersing machine;
e) returning the mixture obtained at the outlet of the dissolution circuit into the vessel through the transferring circuit;
f) adjusting the solution pH with glacial acetic acid et complementing the final volume of the mixture with bi-distilled water;
g) checking the oxygen content of the solution and realizing a desoxygenation by means of white vapor if the oxygen content is superior to 0.2 ppm;
h) transferring the paracetamol solution through a first 0.45 μm-filter then through a second 0.22 μm-filter;
i) realizing the packaging step consisting in filling a flexible pocket with the paracetamol solution filtered at the step h), and in hermetically packaging said pocket into an over-package with an oxygen absorbent; and
the steps a) to h) being realized in said hermetically sealed reactor in an atmosphere desoxygenated with white vapor of bi-distilled water.

6. Method according to claim 5, wherein the flexible pocket containing the paracetamol solution comprises a header space with a limited air volume between 5 and 10 cm³.

7. Method according to claim 5, wherein at the step i) a partial vacuum is realized in said over-package before packaging said flexible pocket and said absorbent with the over-package.

8. Method according to claim 5, wherein the step i) of the method is realized in a packaging room with a cleanliness class A or B in order to avoid the sterilization step in the autoclave.

9. Method according to claim 5, wherein the step i) of the method is realized in a packaging room with a cleanliness class C, the method comprising a supplemental step consisting in an autoclaving sterilization of the packaged solution after step i).

10. Device for packaging an injectable and ready-to-use paracetamol solution according to claim 1, comprising:
a primary package consisting in a flexible pocket composed of at least one compartment, one of the compartments containing said solution,
a secondary package used as a over-package for said pocket and comprising two material walls the edges of which are sealed for ensuring the tightness properties of the over-package,
an oxygen absorbent placed in said over-package, said absorbent being destined to catch the oxygen dissolved in the solution and the oxygen present in the over-package.

11. Device according to claim 10, wherein a partial vacuum is realized in said over-package.

12. Device according to claim 10, wherein said oxygen absorbent is a mixture with iron-oxide-based mineral material.

13. Device according to claim 10, wherein the walls of said over-package are made from a complex material polyester/polyamide/aluminum/polypropylene or a complex material polyester/polypropylene.

14. Device according to claim 13, wherein the walls of said over-package have an oxygen permeability which is at least inferior to 1 cm³/m²/24 h.

15. Device according to claim 10, wherein said flexible pocket is made from a plastic material selected from the group comprising polyvinyl chloride, polypropylene or a polypropylene-based multilayer complex material.

16. Device according to claim 15, wherein the walls of said flexible pocket have an oxygen permeability between 950 cm³/m²/24 h and 1000 cm³/m²/24 h.
